(19) United States
(12) Patent Application Publication
(10) Pub. No.: US 2018/0104174 A1
(43) Pub. Date: Apr. 19, 2018

(54) Cosmetic Use of a Diospyros Mespiliformis Extract Alone or in Combination with Other Active Agents Such as an Anti-Pollutant

(71) Applicant: Laboratoires Clarins, Paris (FR)

(72) Inventors: Olivier Courtin, Neuilly Sur Seine (FR); Richard Fitoussi, Maisons Laffitte (FR); Sandrine Weber, Cormeilles En Vexin (FR)

(73) Assignee: Laboratoires Clarins, Paris (FR)

(21) Appl. No.: 15/567,205

(22) PCT Filed: Apr. 15, 2016
(86) PCT No.: PCT/FR2016/050895

§ 371 Date: Nov. 16, 2017

(30) Foreign Application Priority Data
Apr. 20, 2015 (FR) 1553518

(51) Int. Cl. A61K 8/96; A61Q 17/00

(52) U.S. Cl. CPC A61K 8/96; A61Q 17/00

(57) ABSTRACT

The present invention relates to the cosmetic of a Diospyros mespiliformis extract alone or in combination with a Sanicula europea extract and/or a Furcellaria lumbricalis extract and/or a Lampsana communis extract to fight against and/or prevent the effects of pollution on the skin. The invention also relates to a cosmetic process for the cosmetic prevention or treatment of the effects of pollution on the skin consisting of applying a Diospyros mespiliformis extract, alone or in combination with other active agents, to the skin.

The present invention relates to the cosmetic use of a Diospyros mespiliformis extract alone or in combination with a Sanicula europea extract and/or a Furcellaria lumbricalis extract and/or a Lampsana communis extract for combatting and/or preventing the effects of pollution on the skin; the invention also relates to a cosmetic process for the cosmetic prevention or treatment of the effects of pollution on the skin, consisting in applying a Diospyros mespiliformis extract, alone or in combination with other active agents, to the skin.

Pure air is a mixture of 78% nitrogen, 21% oxygen, 0.09% argon, and 0.1% water vapor and other gases that are more or less rare, including carbon dioxide, hydrogen and ozone.

Atmospheric pollution is defined by the modification of pure air by modification of its components or by the addition of harmful elements. Very schematically, it is possible to consider that the pollutants currently listed are essentially produced by industry, heating and traffic. It is usual to classify pollutants into seven chemical families:
oxidizing agents such as ozone or nitrogen oxides are irritants and free-radical generators;
dust is particles in suspension which sometimes fix polycyclic hydrocarbons; their harmful role is modified by the temperature and the degree of moisture of the air, acid particles are irritant; a drop in hydration and oxygenation of the tissues is observed;
organic chemical products are carcinogenic, they come from industrial waste and from motor vehicle combustion;
carbon monoxide leads to tissue hypoxia, 80% of it comes from motor vehicles;
hydrocarbons and solvents represent 50% of motor vehicle pollution, they are toxic, irritant, carcinogenic and mutagenic; in the presence of light, they react with nitrogen oxides and produce ozone;
sulfur dioxide is one of the products of the combustion of heating or domestic oil and of coal, it causes a modification of the hydro-lipid film of the skin and skin irritations;
metals such as lead, zinc, aluminum, mercury, etc. can interfere with cell metabolism, by affecting enzymatic reactions. They participate in oxidative damage with DNA lesions and cell lipid lesions.

The air inside dwellings, which is in continuity with the outside air, also comprises its own sources of pollutants that may be of biological origin (sources of moisture and of molds, endotoxins, allergens, etc.), physical origin (radon, particles, fibers, etc.) and chemical origin (tobacco smoke, aldehydes, volatile organic compounds—VOCs, metals, etc.).

In 1969, Harry Daniell recognized that smokers look older than non-smokers. He subsequently developed a system for grading wrinkles, which allowed him to analyze the association between smoking and wrinkled skin more objectively; with this system, he was able to validate his first impression. Subsequently, his system was used in various epidemiological studies (Daniell H W. Smoker's wrinkles. A study in the epidemiology of “crow's feet”. Ann Intern Med 1971; 75:873-80).

These epidemiological studies proved that chronic smoking was an important environmental factor involved in premature skin aging (Morita A. Tobacco smoke causes premature skin aging. J Dermatol Sci 2007; 48:169-75). There are many physiopathological mechanisms cited. Dehydration of the stratum corneum by tobacco smoke would partly explain the decrease in the thickness of the horny layer (Freiman A et al. Cutaneous effects of smoking. J Cutan Med Surg 2004; 8:415-23). By releasing thiocyanates, while increasing the amount of carboxyhemoglobin and promoting the secretion of vasopressin, chronic smoking is responsible for a relative chronic ischemia of the dermis, with a decrease in transcutaneous partial pressure of O₂. As a result, the dermal metabolism is modified: dystrophic elastic fibers and proteoglycans accumulate, extracellular matrix proteins are further degraded by metalloproteases, and collagen synthesis is decreased.

The association between smoking and aging of the skin appears to be mediated through a higher expression of MMP-1 and MMP-3 mRNA and also a decrease in collagens I and III (Yin L et al. Skin aging induced by ultraviolet exposure and tobacco smoking: evidence from epidemiological and molecular studies. Photodermatol Photoimmunol Photomed 2001; 17:178-83; Yin L et al. Alterations of extracellular matrix induced by tobacco smoke extract. Arch Dermatol Res 2000; 292:188-94).

Smoking also appears to be associated with an increase in elastose and in telangiectasia (Kennedy C et al. Effect of smoking and sun on the aging skin J Invest Dermatol 2003; 120:548-54) indicating other molecular pathways in addition to the induction of MMP-1 expression. It has recently been suggested that the AhR pathway may play a role in the process of premature skin aging induced by tobacco (Morita A et al. Molecular basis of tobacco smoke-induced premature skin aging. J Investig Dermatol Symp Proc 2009; 14:53-5).

The skin, an organ of which the first function is to be an external barrier, is in direct contact with the various atmospheric pollutants and, consequently, the contact of the skin with atmospheric pollution induces in particular aging of the skin.

Some studies have studied the effect of ozone on the skin or on the skin cells. Ozone is a pollutant that can form at ground level following an interaction between solar radiation and exhaust pipe gas emissions. McCarthy et al. have exposed normal human epidermal keratinocytes (NHEKs) to ozone concentrations measured in cities and analyzed their effects. The levels of hydrogen peroxide and of IL-la increase, while the levels of ATP decrease. Finally, McCarthy et al. have noted that ozone increases DNA damage (McCarthy J T et al. Effects of ozone in normal human epidermal keratinocytes. Exp Dermatol. 2013 May 22(5):360-1). Other studies have studied the effect of ozone on murine skin tissue which shows that ozone, a powerful oxidizing agent, is capable of affecting skin integrity (Thiele J J et al. Ozone-exposure depletes vitamin E and induces lipid peroxidation in murine stratum corneum. J Invest Dermatol 1997; 108:753-7). Likewise, a study has shown that ozone is capable of inducing MMP-9 expression in murine skin, indicating a role in matrix remodeling (Valacchi G et al. Induction of stress proteins and MMP-9 by 0.8 ppm of ozone in murine skin. Biochem Biophys Res Commun 2003; 305:741-6).

A recent epidemiological study has discovered a direct link between exposure to particulate matter (PM) and the appearance of signs of skin aging such as pigment spots, but also wrinkles (Vierkötter A et al. Airborne particle exposure and extrinsic skin aging. J Invest Dermatol 2010; 130:2719-26). An important mechanism by which ambient particles exert these harmful effects is the generation of ROSs (Donaldson K et al. Combustion-derived nanoparticles: a review of their toxicology following inhalation exposure. Part Fibre Toxicol 2005; 2:10).

In addition, particles can act as supports for organic chemical products and metals which are capable of localizing in the mitochondria so as to generate therein ROSs which lead to skin aging via mitochondrial lesions (Li N, Sioutas C et al. Ultrafine particulate pollutants induce oxidative stress and mitochondrial damage. Environ Health Perspect 2003; 111:455-60). Another pathway which induces skin aging could be that of polycyclic aromatic hydrocarbons (PAHs). Adsorbed at the surface of particulate matter in the air in urban regions, PAHs are able to activate the metabolism of xenobiotics via AhR (Menichini E. Urban air pollution by polycyclic aromatic hydrocarbons: levels and sources of variability. Sci Total Environ 1992; 116:109-35).

Other studies carried out by Ushio et al. (1999) have tested the action of diesel particles on keratinocytes in culture. High concentrations of diesel particles are responsible for impairments to the growth of these cells and their migration.

Particles therefore appear to have a direct action on skin integrity. Furthermore, this exposure causes an increase in IL-8 secretion. As it happens, this cytokine is involved in inflammatory skin reactions. This study appears to suggest that diesel particles could play a role in the initiation and/or pathogenesis of inflammatory skin disorders such as eczema, allergic dermatitis or psoriasis (Ushio, H., K. Nohara, et al. (1999). “Effect of environmental pollutants on the production of pro-inflammatory cytokines by normal human dermal keratinocytes.” Toxicol Lett 105 (1): 17-24).

It thus appears that exterior and interior atmospheric pollutants are the cause of numerous unattractive skin manifestations: in particular an alteration of the thickness, of the elasticity, of the firmness or else of the radiance of the skin, the appearance of wrinkles and of a dull complexion, or even of pigment spots. Even if these skin manifestations remain superficial and are not pathological, most individuals are more or less exposed to pollutants and there is a strong need to develop novel cosmetic formulations that would make it possible to prevent or curb their appearance.

It is recalled that the skin is a very widespread organ, covering the entire surface of the body, and in total, in adult human beings, covers a surface area of about 1.6 square meters. Its function is to protect the deep tissues from the outside environment and it has an activity in particular on immunity, temperature regulation and fluid loss. The skin consists of three main parts. A thin superficial part called the epidermis, an internal thick part called the dermis, and a deep fatty part called the hypodermis. The epidermis is characterized essentially by its organization in the form of strata corresponding to an increasing state of differentiation of the keratinocytes, which are the cells that constitute approximately 90% of the epidermis. These keratinocytes migrate from the deep layers of the epidermis to the external surface of the epidermis. During this migration, keratinocytes undergo numerous biochemical and structural modifications, the most significant of which is keratinization, a process by which the cells synthesize keratin. The horny layer, which is the most superficial layer of the epidermis, is composed mainly of dead cells called corneocytes and is in particular very resistant to external attacks. The epidermis can be defined as a coating epithelium which constitutes the external structure of the skin. It provides the skin with protection, in particular by the production of keratin, which is a strong filamentous protein. Keratin is partly responsible for the impermeability of the skin.

The dermis is separated from the epidermis by the “dermoepidermal junction”. It is continued, deep down, without clear limit, by the hypodermis. The role of the dermis is in particular to maintain the suppleness and strength of the skin. It also performs other essential functions. Its considerable vascularization allows it to perform nutritive exchanges with the epidermis. It also allows it to be rich in immune cells of macrophage and dendritic cell type, which are essential components of the immune defense of the skin. Furthermore, it is an innervated tissue which has sensory receptors responsible for the sense of touch. Finally, it is involved in the phenomena of healing, and of the regulation of keratinocyte proliferation and differentiation by synthesizing soluble cytokines and growth factors intended for the epidermis. The dermis is a connective support tissue essentially consisting of fibroblasts and of a microfibrillar network of collagen, elastin and proteoglycans forming the extracellular matrix. The extracellular matrix is the adhesive substrate of fibroblasts and the mechanical support of the tissue.

In the context of research aimed at identifying active agents intended for protecting and/or repairing the effects of exterior and/or interior atmospheric pollution on the skin, the applicant has discovered that Diospyros mespiliformis extracts have a marked activity against the effects of formaldehyde, of cigarette smoke and of fine particles on the skin (see the tests described in example 1).

The present invention thus relates to the cosmetic use of a Diospyros mespiliformis extract or to a cosmetic composition comprising the same, for preventing and/or repairing the effects of pollution on the skin; the use according to the invention is particularly suitable for application to healthy skin.

In the context of the present invention, the term “healthy skin” denotes skin which does not exhibit any skin pathology and which, in addition, is in good condition (under article 2.1.a) of (EC) REGULATION No. 1223/2009 OF THE EUROPEAN PARLIAMENT AND OF THE COUNCIL of Nov. 30, 2009, relating to cosmetic products).

Diospyros mespiliformis, also called African ebony, is a plant of the family Ebenaceae, it is an evergreen tree that originates in Africa and that can reach a height of 40 cm with a very branched crown and dense foliage. The leaves alternate and are simple and whole and the flowers are white to greenish-yellow. Its fruit is traditionally consumed as a drink or as a broth mixed with cornflower.

This plant is used for the preparation of traditional remedies, in particular owing to its antibiotic properties; it is also used for improving fertility, treating malaria, syphilis, leprosy or mycoses, or else as an anthelmintic. Leaf decoctions are used to treat fever, ear infections and wounds.

To date, no activity of this plant in preventing or repairing the effects of pollution on the skin has been described.

The tests carried out, on models of healthy epidermis, treated or not treated with a Diospyros mespiliformis extract and exposed to various pollutants, by the applicant have made it possible to demonstrate the following effects of the Diospyros mespiliformis extract:
This extract prevents and corrects the harmful effects of formaldehyde on the skin by reinforcing the skin barrier through the induction of the expression of the constituent markers of the horny envelope (hornerin HRNR, Small Proline Rich Protein SPRR2A, CE2B, LCE3D); this effect is also confirmed by the identification, by immunohistochemistry, of the induction of the expression of loricrin, a major protein of the horny envelope. In addition, this extract also induces increased expression of SIRT1, a protein involved in the homeostasis of keratinocyte proliferation and differentiation processes. Finally, the Diospyros mespiliformis extract protects the epidermis subjected to formaldehyde by reducing the inflammatory manifestations; indeed, the results obtained show that the markers of inflammation, of apoptosis and of matrix degradation are underexpressed; the assaying of the cytokine IL-8 and of the matrix proteases MMP1 and MMP9 confirm the results of the transcriptomic study.
The Diospyros mespiliformis extract protects the epidermis subjected to cigarette smoke by no longer soliciting the elements involved in inflammation. The results of the tests presented in example 1 show that the markers of inflammation, of apoptosis and of matrix degradation are underexpressed in the presence of the extract; the assaying of the cytokines IL1-α and IL-8 and of MMP1 confirm the results of the transcriptomic study. In addition, the Diospyros mespiliformis extract makes it possible to ensure that good moisturization is maintained (FLG2, CD44). The results obtained again show that the use of this extract on the epidermis makes it possible to preserve and restore cell cohesion and cell communication provided in particular by claudin 1 (CLDN1) and desmocollin 1 (DSC1) and also the dermoepidermal junction against the effects of cigarette smoke (maintaining or restoring collagen IV and VII gene expression). These tests also show that the Diospyros mespiliformis extract contributes to maintaining skin homeostasis when said skin is exposed to pollutants.
Finally, the Diospyros mespiliformis extract prevents and repairs the modification of the barrier function by atmospheric particles; this results from the strong increase in the expression of loricrin which plays a key role in the skin barrier function. The tests carried out by comparison of a healthy epidermis modified by the presence of particles and of an epidermis treated with a Diospyros mespiliformis extract before being exposed to particles show that the latter preserves good cell cohesion and good cell communication ensured in particular by claudin 1 (CLDN1) and desmocollin 1 (DSC1) and has its dermoepidermal junction protected against the effects of the particles (increase in the expression of the genes of the major components of the dermoepidermal junction, collagens IV, VII and nidogen I).

The Diospyros mespiliformis extract that can be used according to the invention can come from any part of the plant; preferably, it is the aerial parts of the plant and more preferentially the leaves. The extraction of the extract from the plant or from the parts chosen can be carried out by milling and extraction with aqueous and/or organic solvents, preferably aqueous solvents.

According to one particular embodiment of the invention, the Diospyros mespiliformis extract used according to the invention is an aqueous leaf extract stabilized with maltodextrin, obtained by means of the following steps:
milling of Diospyros mespiliformis leaves;
extraction with water;
separation;
purification;
addition of maltodextrin;
filtration;
spray-drying.

The extract thus obtained is a powder that is yellow-orange to beige in color with a characteristic odor; prepared in aqueous solution at 1%, its pH is between 5.0 and 7.0.

Preferably, the Diospyros mespiliformis extract is formulated in a cosmetic composition, the composition of which those skilled in the art will know how to adapt to the physicochemical properties of the extract.

Preferably, said cosmetic composition comprises from 0.01% to 10%, preferably from 0.01% to 2%, of Diospyros mespiliformis extract by weight of solids relative to the total weight of the composition.

Conventionally, this composition may further comprise one or more formulating agents or additives of known use and used in cosmetic and dermatological compositions, such as, by way of examples and without limitation, softeners, dyes, film-forming active agents, surfactants, fragrances, preservatives, emulsifiers, oils, glycols, vitamins such as vitamin E, UV-screening agents, etc. By virtue of this knowledge about cosmetics, those skilled in the art will know which formulating agents to add to the compositions of the invention and in what amounts depending on the properties desired.

This composition can be in any form known to those skilled in the art in the cosmetology and dermatology field without any galenical restriction other than the application to the face or to the body. Advantageously, it is in the form of a gel, a lotion, a cream, an emulsion, a milk, a spray, etc.

The present invention thus relates to the cosmetic use of a Diospyros mespiliformis extract or of a composition comprising same, for protecting the skin against damaging effects on the skin, that is to say unwanted visible manifestations on the skin, caused by atmospheric pollution, and/or repairing these skin manifestations or disorders caused by atmospheric pollution; the use according to the invention is advantageous whether the atmospheric pollution results from the presence of interior or exterior pollutants; it may in particular involve pollution resulting from the presence in the surrounding air of oxidizing agents such as ozone or nitrogen oxides, of carbon monoxide, of hydrocarbons and solvents, of sulfur dioxide, of metals, of formaldehyde, of cigarette smoke, of particulate matter or fine dust in suspension, or else of electromagnetic waves.

More particularly, the use of a Diospyros mespiliformis extract or of a composition comprising same makes it possible to prevent the appearance of visible cutaneous manifestations on the skin, or signs of degradation thereof, and/or to repair skin disorders caused by pollution, by means of the following activities:
increase in the cohesion of the skin and of the stratum corneum and improvement in the barrier function of the skin;
restoration of the homeostasis of the keratinocyte proliferation and differentiation processes;
restoration of the dermal matrix;
restoration of the moisturization of the skin;
improvement of the cohesion of the skin cells;
improvement of the cell communication;
restoration of the dermoepidermal junction.

On the basis of these properties, the use of the Diospyros mespiliformis extract or of a composition comprising same is particularly useful for preventing and/or limiting and/or correcting skin aging caused by pollution and the various skin manifestations that are associated therewith, in particular, preventing and/or correcting wrinkles and fine lines and/or preserving the radiance and/or increasing the moisturization and/or regulating the desquamation and/or maintaining or stimulating the suppleness of the skin subjected to pollution.

In order to broaden the spectrum of action of the Diospyros mespiliformis extract or of the composition comprising same, it can be advantageous to combine it with other cosmetic active agents which strengthen its cosmetic effects and/or have one or more other complementary cosmetic effects.

It is thus advantageous to combine the Diospyros mespiliformis extract, optionally in a cosmetic composition with one or more other antioxidant and/or anti-pollution cosmetic agents.

Preferably, these cosmetic agents are chosen from:
a Sanicula europea extract which has anti-oxidizing properties (cf. example 2) and which also has the property of reinforcing the stratum corneum and thus the barrier function of the skin (FR 3 007 980). This extract thus makes it possible to reinforce both the preventive action against pollution and also the repair of the skin by the Diospyros mespiliformis extract.

Sanicula europea is a herbaceous plant of the family Apiaceae (Umbellifers) which generally grow in the undergrowth.

It is a glabrous perennial plant of medium size (15-50 cm) which can be easily recognized through the globular appearance of its umbels (3 to 5 unequal ribs).

It is known to be rich in saponins, tanins and mucilages; it is used in traditional medicine for its healing, astringent and wound-healing properties.

The sanicle extract is preferentially a water-soluble extract of the aerial parts (leaves, flowers and stems). Advantageously, this extract is an aqueous-alcoholic extract of the aerial parts and it is preferably stabilized with glycerol.

Preferably, it is an aqueous-alcoholic extract obtained according to a process comprising at least the following steps:
drying of the aerial parts;
milling;
ethanol/water (80:20) extraction;
filtration;
concentration under vacuum by evaporation of the extraction solvents;
adjustment of the concentration (addition of glycerol).

The Sanicula Europaea extract is a yellow-brown slightly viscous liquid with a characteristic odor. It has the following analytical characteristics: pH=3.5-5.0; relative density: 1.050-1.200; refractive index: 1.370-1.400; dry plant/extract ratio=1/10.

Preferably, the cosmetic composition used according to the invention comprises from 0.01% to 10% of Sanicula Europaea extract by weight of solids relative to the total weight of the composition;
an extract of Furcellaria lumbricalis, which is a red alga from the Baltic Sea. Preferably, the extract comprises oligofurcellaran enriched with marine salts. Oligofurcellaran is obtained by depolymerization of a sulfated furcellaran (galactose and anhydrogalactose) extracted from the alga; following the depolymerization, the oligofurcellaran is rehydrated with a solution enriched with marine salts, such a type of extract is sold by the company Codif under the name Hydranov P.

This Furcellaria lumbricalis extract has skin-moisturizing properties and it makes it possible to prevent or correct skin dehydration due to pollution (see example 3).

Preferably, said cosmetic composition comprises from 0.01% to 10%, preferably from 0.1% to 2%, of Furcellaria lumbricalis extract by weight of solids relative to the total weight of the composition;
a Lampsana communis extract; Lampsana communis, of the family Asteraceae, is a very widespread annual plant, which grows at the edge of roads and other sites subjected to atmospheric pollution. It is an undemanding and very resistant plant.

The Lampsana communis extract used in the composition according to the invention is obtained by aqueous-glycolic extraction of the aerial part of the plant. A clear, brown-colored liquid with a characteristic odor is thus obtained, having the following analytical characteristics:
water content: 18 to 26%
pH: 6 to 8
density: 1.055 to 1.075
refractive index: 1.420 to 1.440.

When this extract is used, it is added at a concentration of about from 0.01% to 5% by weight, preferably 0.01% to 0.5% by weight and most preferentially from 0.01% to 0.05% by weight.

The present invention also relates to the use of one of the following combinations or to a composition comprising same:
a Diospyros mespiliformis extract and a Sanicula europea extract;
a Diospyros mespiliformis extract and a Furcellaria lumbricalis extract;
a Diospyros mespiliformis extract and a Lampsana communis extract;
a Diospyros mespiliformis extract, a Sanicula europea extract and a Furcellaria lumbricalis extract;
a Diospyros mespiliformis extract, a Sanicula europea extract and a Lampsana communis extract;
a Diospyros mespiliformis extract, a Furcellaria lumbricalis extract and a Lampsana communis extract;
a Diospyros mespiliformis extract, a Sanicula europea extract, a Furcellaria lumbricalis extract and a Lampsana communis extract;
   for combating and/or preventing the effects of pollution on the skin, in particular for protecting the skin against the harmful effects on the skin of atmospheric pollution and/or repairing skin disorders, that is to say the unwanted visible manifestations on the skin, caused by atmospheric pollution; the use according to the invention is advantageous whether the atmospheric pollution results from the presence of interior or exterior pollutants; it may in particular be pollution resulting from the presence in the surrounding air of oxidizing agents such as ozone or nitrogen oxides, of carbon monoxide, of hydrocarbons and solvents, of sulfur dioxide, of metals, of formaldehyde, of cigarette smoke, of particulate matter or fine dust in suspension, of electromagnetic waves.

More particularly, the use of one of the combinations according to the invention or of a composition comprising same makes it possible to prevent the appearance of the visible cutaneous manifestations on the skin or signs of degradation thereof and/or to repair these skin manifestations or disorders caused by pollution, by means of the following activities:
increase in the cohesion of the skin and of the stratum corneum and improvement in the barrier function of the skin;
restoration of the homeostasis of keratinocyte proliferation and differentiation processes;
restoration of the dermal matrix;
restoration of the moisturization of the skin;
improvement in the cohesion of the skin cells;
improvement in the cell communication;
restoration of the dermoepidermal junction.

On the basis of these properties, the use of one of the combinations according to the invention or of a composition comprising same is particularly useful for preventing and/or limiting and/or correcting skin aging caused by pollution and the skin manifestations that are associated therewith, in particular preventing and/or correcting wrinkles and fine lines and/or preserving the radiance and/or increasing the moisturization and/or regulating the desquamation and/or maintaining or stimulating the suppleness of the skin subjected to pollution.

Finally, the present invention relates to a cosmetic care process for preventing and/or repairing the effects of pollution on the skin, characterized in that an appropriate amount of a Diospyros mespiliformis extract, alone or in combination with a Sanicula europea extract and/or a Furcellaria lumbricalis extract and/or a Lampsana communis extract or of a composition comprising said extract or one of said combinations, is applied to the skin.

### FIGURES

FIG. 1 represents the Vitrocell® exposure model used in the formaldehyde model.

The results of the proteomic studies carried out on a model of epidermis treated with a Diospyros mespiliformis extract, exposed to formaldehyde, are presented in FIGS. 2A and 2B.

The results of the proteomic studies carried out on a model of epidermis treated with a Diospyros mespiliformis extract, exposed to tobacco smoke, are presented in FIGS. 3A and 3B.

The results of the proteomic studies carried out on a model of epidermis treated with a Diospyros mespiliformis extract, exposed to fine particles, are presented in FIGS. 4A and 4B.

FIG. 5 is a histogram representing the effect on the level of mitochondrial ROSs of keratinocytes exposed to various conditions (nontreated-cell control, H₂O₂, resveratrol and Sanicle extract at various concentrations).

FIG. 6 is a histogram representing the effect on the level of cellular ROSs of keratinocytes exposed to various conditions (nontreated-cell control, H₂O₂, resveratrol and Sanicle extract at various concentrations).

FIG. 7 is a histogram representing the effect on the activity of cellular SOD of keratinocytes exposed to various conditions (nontreated-cell control, H₂O₂, resveratrol and Sanicle extract at various concentrations).

FIG. 8 is a histogram representing the effect on the activity of mitochondrial SOD of keratinocytes exposed to various conditions (nontreated-cell control, H₂O₂, resveratrol and Sanicle extract at various concentrations).

FIG. 9 is a histogram representing the effect on the level of total glutathione of keratinocytes exposed to various conditions (nontreated-cell control, H₂O₂, resveratrol and Sanicle extract at various concentrations).

FIG. 10 is a histogram representing the degree of cutaneous moisturization of skin explants exposed to exhaust gas pollution and treated or not with a Furcellaria lumbricalis extract.

### EXAMPLE 1

The demonstration of the anti-pollution activity of the Diospyros mespiliformis extract was enabled by the development of novel models capable of characterizing the effects of various pollutants of the interior and exterior atmosphere during single or repeated exposure on the biological response of reconstituted epithelia in vitro.

Briefly, the study model chosen is the RHE reconstructed epidermis (SkinEthic Lyon, France). It is an epidermis reconstituted from primary human cells. The keratinocytes are cultured, in air/liquid interface, in a serum-free defined medium on inserts having a polycarbonate membrane (Nunc insert 0.5 cm²).

The setting up of a reconstructed-epidermis air-liquid-interface exposure model makes it possible to study the interactions between skin epithelial cells, in an environment close to the in vivo situation, and environmental pollutants.

Three types of pollutants were applied to the surface of the epidermis: formaldehyde, tobacco smoke, particles.

### Formaldehyde Model

Formaldehyde, better known as formol when it is dissolved in water, is a very volatile organic compound (VOC) belonging to the aldehyde family. This substance has a low molecular weight and has the property of becoming gaseous at ambient temperature and is currently frequently found in interior environments.

Formaldehyde has a very large number of sources, among which are: combustion sources encompassing tobacco smoke, candles, incense sticks, fireplaces and also gas cookers, oil-burning stoves, and construction and decorating products containing formaldehyde-based components.

For the study, the reconstructed epidermis model was exposed to a formaldehyde dose of 2100 ppm.

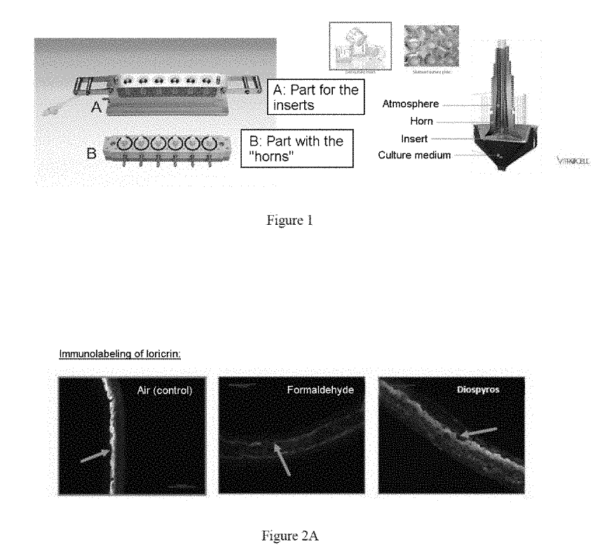

The exposures are carried out in a six-well Vitrocell© exposure module suitable for the samples. This module is made up of two parts providing an air/liquid interface with a contact (i) between the basal part of the epidermises and the nutritive medium and (ii) between the apical part and the atmosphere generated (air or pollutant(s)) (FIG. 1):
the part A which receives the inserts (the epidermises). The maintenance culture medium (MM) is deposited in the lower part of the module that is temperature-regulated (37° C.) by means of water circulation,
the direct contact between the gaseous atmosphere and tissue surface is provided by the part B of the exposure module and pollutant admission horns.

Various parameters, such as the air flow rate, the sample exposure time and the exposure frequency, were defined with a “control” atmosphere, such as air. The objective of this step was to define the best conditions for air/liquid interface exposures without the tissue suffering.

### Tobacco Smoke Model

Tobacco smoke, which is a mixture containing aldehydes, including formaldehyde and acetaldehyde, and a large number of volatile organic compounds, was generated from a commercial cigarette. 10×20 ml were taken up with a syringe and introduced into an exposure chamber. The epidermises were exposed to 200 ml of tobacco smoke for 60 minutes at 37° C. An “air-control” was studied in parallel.

### Fine Particles Model

Fine particles come from road traffic, more particularly diesel engines of motor vehicles, but also from industry, agriculture and combustion (chimneys, individual heating, etc.). Microparticles, which are micrometric in size, are not visible to the naked eye. It is these that are measured in the air through the PM10 particles (size less than 10 μm, 6 to 8 times smaller than the size of a cell) and the fine or PM2.5 particles (size less than 2.5 μm, such as bacteria).

For the study, the particles (composition: table below) were applied topically to the surface of the reconstructed tissue. The epidermises were exposed to the particles for 60 minutes at 37° C. An “air-control” was studied in parallel.

Organic compounds in the two air pollution PM samples.

Concentration (ng/in³)

| | PM_{2.5} | PM_{2.3} |
|---|---|---|
| VOCs | | |
| Benzena | 0.45 | 0.05 |
| Toluene | 1.81 | 0.07 |
| Ethylbenzene | 0.83 | 0.10 |
| e-Xylene | 3.47 | 0.07 |
| 1 2 3 Trimethylbenzene | 1.17 | 0.71 |
| Trimethylbenzene | 0.52 | <LQ |
| Tetramethylbenzene | 0.35 | 0.20 |
| Pentamethylbenzene | 0.29 | <LQ |
| Total | 8.90 | 1.2 |

| | | | |
|---|---|---|---|
| PAHs | | | |
| Naphralene | Nap | 0.01 | 0.01 |
| Aceaxphtylene | Ace | <LQ | 0.004 |
| Phenanthrene | Phe | 0.02 | 0.01 |
| Fluoranthene | Flu | 0.11 | 0.03 |
| Pyrene | Pyr | 0.28 | 0.06 |
| Benzo[a] [symbol] | BaA | 0.19 | <LQ |
| Chrysene | Chr | 0.26 | 0.01 |
| Benzo[b]Fluoranthene | BbF | <LQ | 0.02 |
| Benzo[k]Fluoranthene | BkF | 0.30 | <LQ |
| Benzo[a]Pyrene | BaP | 0.24 | 0.01 |
| [symbol] [a b]Anthracene | DahA | 0.21 | <LQ |
| Indeno[1 2 3-cd]Pyrane | Ind | <LQ | 0.07 |
| Benzo[g b j]Perylene | BghrP | 0.37 | <LQ |
| Total | | 1.99 | 0.22 |

<LQ = Under limit of quantification;
VOCs = Volatile Organic Compounds,
PAHs = Polycyclic Aromatic Hydrocarbons [symbol]
indicates data missing or illegible when filed

Various methods of investigation made it possible to characterize the impact of these pollutants on the Skinethic® reconstructed epidermis model:
a transcriptomic method carried out by quantitative PCR (126 “epidermal” genes);
a proteomic method carried out by immunohistochemistry;
a method for assaying the culture media using the Elisa technique and the multiplex technique.

In the context of the evaluation of the protective effect of the Diospyros mespiliformis extract with respect to functional and structural modifications of the epidermis that are induced by the pollutants (formaldehyde, cigarette smoke, particles), the following tests were carried out:

The Skinethic® reconstructed epidermis is treated with each of the pollutants, combined or not combined with a Diospyros mespiliformis extract applied to the surface of the tissue.

The Diospyros mespiliformis extract tested is an aqueous leaf extract stabilized with maltodextrin, obtained by means of the following steps:
milling of Diospyros mespiliformis leaves;
extraction with water;
separation;
purification;
addition of maltodextrin;
filtration;
spray-drying.

Characterization: powder that is yellow-orange to beige in color with a characteristic odor; prepared in aqueous solution at 1%, its pH is between 5.0 and 7.0.

At the end of the treatments, each epidermis is preserved in order to carry out gene expression analyses by RTqPCR and epidermal-target immunolabelings. The culture media are used for the Elisa and multiplex assays.

### Transcriptomic Study:

The transcriptomic study is carried out by quantitative PCR (qPCR) on the Skinethic® reconstructed epidermises, making it possible to study the expression of numerous genes. The RNAs are extracted using RLT buffer and then a Qiagen RNeasy extraction microkit; they are subsequently assayed and then reverse transcribed. The qPCR is then carried out on plates 128 epidermal function genes (Roche Diagnostics).

The results are analyzed by calculating the difference in expression between two conditions, called Fold Change (FC). It is considered that an increase in expression relative to the control is reflected by an FC≥1.5; and that a decrease in expression relative to the control is reflected by an FC≤0.7.

### Proteomic Study:

The proteomic study is carried out by immunohistochemistry, making it possible to study the presence of numerous proteins.

The reconstructed epidermises are fixed in 10% formalin for 72 h, then dehydrated and embedded in paraffin blocks, before being cut into 5 μm sections. The sections are deparaffinized and then unmasked (Abcam) before being treated with 0.1% triton X-100 (Sigma) and 2% bovine serum albumin (BSA—Sigma) for 10 minutes, in order to permeabilize the membrane and saturate the binding sites.

The sections are then incubated for 1 h with the primary antibodies directed against loricrin and 4-HNE (4-hydroxynonenal).

The photographs are taken using a fluorescence conversion microscope (Nikon Eclipse 50i).

### Assaying by Elisa, by Multiplexing (Luminex Technology):

ELISA (acronym for Enzyme Linked ImmunoSorbent Assay) is an immunological assay intended to detect and/or assay a protein in a biological liquid. In the “sandwich” assaying technique, the wells of a microplate are coated with a capture antibody capable of specifically binding the antigen being sought. During this operation, called coating, the capture antibody binds to the plastic of the wells by electrostatic interaction. A second antibody, the trace antibody, capable of binding to the captured antigen, is then added to the wells and the unbound tracer antibodies are removed by rinsing. The tracer antibody is coupled to an enzyme catalyzing the formation of a colored product. The reaction can thus be quantified by colorimetry on the basis of a calibration curve produced with known concentrations of antigen, since the number of molecules of tracer antibody bound depends on the number of antigen molecules immobilized by the capture antibody. This assay is used for detecting the presence of IL-1α and of IL-8.

The Luminex technology, based on the principle of an ELISA combined with flow cytometry, makes it possible to simultaneously assay numerous protein or nucleic targets in small sample volumes. The technology employs the use of microspheres which are either polystyrene beads or magnetic beads that are colored with a mixture of two fluorochromes, the ratio which is known and perfectly defined for each type of bead.

Once the molecules of interest are bound to the surface of the beads, the latter are incubated with the samples to be analyzed. After having followed an experimental protocol of ELISA type, the results are obtained by reading on a Luminex which, by virtue of a fluid pipetting and management module, suctions the samples containing the beads in suspension into a liquid vein in such a way that they are one after the after (analogy with flow cytometry) and then excited by a set of two lasers:
a red laser for determining the color code of each bead,
a green laser which excites a fluorochrome coupled to a reporter molecule which makes it possible to detect and assay the antigen-antibody interaction.

This test is used for detecting the presence of MMP1, MMP3 and MMP9.

### Results

Assays with Formaldehyde
Transcriptomic Study:

The results of the proteomic studies are presented in FIGS. 2A and 2B.

The Diospyros mespiliformis extract exerts a protective effect on the skin barrier by increasing the expression of the constituent markers of the horny envelope (hornerin HRNR, Small Proline Rich Protein SPRR2A, LCE2B, LCE3D). These results are confirmed with the identification by immunohistochemistry of loricrin, a major protein of the horny envelope. Likewise, the expression of SIRT1 involved in the homeostatis of keratinocyte proliferation and differentiation processes is substantially re-increased.

The Diospyros mespiliformis extract protects the epidermis subjected to formaldehyde by no longer soliciting the components of inflammation. The results show that the inflammation, apoptosis and matrix degradation markers are underexpressed in the presence of Diospyros mespiliformis extract under the chosen experimental conditions. The assaying of the IL8 cytokine and of the MMP1 and MMP9 matrix proteases confirm the results of the transcriptomic study.

Assays with Cigarette Smoke
Transcriptomic Study:

The results of the proteomic studies are presented in FIGS. 3A and 3B.

The Diospyros mespiliformis extract protects the epidermis subjected to cigarette smoke by no longer soliciting the components of inflammation. The results show that the inflammation, apoptosis and matrix degradation markers are underexpressed in the presence of the extract under the chosen experimental conditions. The assaying of the IL la and IL8 cytokines and of MMP1 confirm the results of the transcriptomic study.

The Diospyros mespiliformis extract also makes it possible to ensure that a good level of moisturization is maintained (FLG2, CD44).

The results obtained by treatment of the epidermis with the Diospyros mespiliformis extract maintain a good preservation of cell cohesion and of cell communication provided in particular by claudin 1 (CLDN1) and desmocolin 1 (DSC1).

The Diospyros mespiliformis extract preserves the dermoepidermal junction against the effects of cigarette smoke. The expression of the genes for collagens IV and VII is ensured.

Assays with the Particles
Transcriptomic Study:

The results of the proteomic studies are presented in FIGS. 4A and 4B.

The Diospyros mespiliformis extract protects the barrier function of the skin against the particles. In particular, the expression of loricrin which has a key role in the barrier function, is greatly increased.

The results obtained by treatment of the epidermis with the Diospyros mespiliformis extract show the conservation of cell cohesion and of cell communication provided in particular by claudin 1 (CLDN1) and desmocollin 1 (DSC1).

The Diospyros mespiliformis extract tends to protect the dermoepidermal junction against the effects of the particles. The expression of the genes of the major components of the EDJ (collagens IV, VII and nidogen I) is increased.

### EXAMPLE 2

The objective of this study is the characterization of the antioxidant property of a Sanicula europea extract by evaluating the parameters of the redox state after induction of an oxidative stress by hydrogen peroxide (H₂O₂) in primary cultures of normal human keratinocytes.

The extract tested is an aqueous-alcoholic extract obtained as follows:
drying of the aerial parts;
milling;
ethanol/water (80:20) extraction;
filtration;
concentration under vacuum by evaporation of the extraction solvents;
adjustment of the concentration (addition of glycerol).

Characterization: yellow-brown, slightly viscous liquid with a characteristic odor. pH=3.5-5.0; relative density: 1.050-1.200; refractive index: 1.370-1.400; dry plant/extract ratio=1/10.

The Mitosafe® technology makes it possible to explore the antioxidant effects of active agents at the mitochondrial level. This technology consists of the integrated study of mitochondrial functions in living cells by multiplexed measurement of parameters such as cell viability, production of cell and mitochondrial oxygen free radicals, evaluation of antioxidant defenses. These tests in 96-well plates are carried out on primary cultures of human epidermal cells.

The following were measured:
mitochondrial reactive oxygen species;
ROS cell production levels;
mitochondrial and cell superoxide dismutase (SOD);
total glutathione (oxidized and reduced form).

In the keratinocytes, exposure to H₂O₂ increases mitochondrial ROS production, which leads to an overregulation of mitochondrial SOD activity and intracellular accumulation of ROSs. It is also responsible for the increase in total glutathione level, in response to the intracellular increase in ROSs. The up-regulation of the mitochondrial SOD activity and the glutathione level are compensatory mechanisms which counteract the oxidative stress induced by ROS accumulation in the intracellular space. Cell SOD is also a direct antioxidant mechanism. Nevertheless, in this model, it is not induced by exposure to H₂O₂.

Antioxidant Property through the Production of Cell ROSs:

The Mitoread AntiOx cROS methodology consists of the quantification of the antioxidant activity of the ingredients toward cell reactive oxygen species (ROSs) using a fluorescent label.

First of all, the cells were pre-incubated with the fluorescent probe at 37° C. for 30 minutes. After washing, the cells were co-incubated with the antioxidant reference compound, 10 Mm Trolox, or the extracts, at concentrations determined to be non-cytotoxic, in the presence of the pro-oxidizing reference compound, 100 μM H₂O₂. The cell ROS production was immediately recorded for 60 minutes. The fluorescence is recorded every five minutes using a microplate reader (Varioskan-Thermo). The cells were maintained at 37° C. during the kinetics. All the extracts were tested in quadruplicate. Their effects were simultaneously compared with the negative control (H₂O₂) and positive control (Trolox).

Antioxidant Property through Mitochondrial ROS Production:

The Mitoread AntiOx mtROS methodology consists of the quantification of the antioxidant activity of the ingredients toward mitochondrial reactive oxygen species (ROSs) using a fluorescent label.

The cells were pre-incubated at 37° C. for 60 minutes with the reference antioxidant, resveratrol 1 μM, or the active ingredients at the predetermined concentrations.

Next, they were incubated with the fluorescent probe at 37° C. for 30 minutes, as a replacement for the antioxidant compounds. After washing, the cells were treated with the reference pro-oxidizing compound, hydrogen peroxide H₂O₂ at 100 μM. The mitochrondrial ROS production was immediately recorded for 60 minutes. The fluorescence is recorded every five minutes using a microplate reader (Varioskan-Thermo). The cells were maintained at 37° C. during the kinetics. All the extracts were tested in quadruplicate. Their effects were simultaneously compared with the negative control (H₂O₂) and positive control (resveratrol).

Antioxidant Property through Cell and Mitochondrial SuperOxide Dismtatase Activities:

The Mitoread RedOx c/mtSOD methodology consists of the quantification of the cell and mitochondrial SOD activities.

The cells were pre-incubated at 37° C. for 60 minutes with the reference antioxidant, resveratrol at 1 μM, or the active ingredients at the predetermined concentrations. The cells were treated with the reference pro-oxidizing compound, hydrogen peroxide H₂O₂ at 100 μM for 60 minutes. Immediately after, the cell extracts were prepared and stored at −80° C. The enzymatic activities were measured by means of a fluorometric and enzymatic method using a microplate reader (Varioskan-Thermo). All the extracts were tested in triplicate. Their effects were simultaneously compared with the negative control (H₂O₂) and positive control (resveratrol).

Antioxidant Property through the Total Glutathione Level:

The Mitoread RedOx GSHtot methodology consists of the quantification of the total glutathione level (GSH+GSSG).

The cells were pre-incubated at 37° C. for 60 minutes with the reference antioxidant, resveratrol 1 μM, or the extract at the predetermined concentrations. The cells were treated with the reference pro-oxidizing compound, hydrogen peroxide H₂O₂ at 100 μM, for 60 minutes. Immediately after, the cell extracts were prepared and stored at −80° C. The total glutathione level was measured by means of a fluorometric and enzymatic method using a microplate reader (Varioskan-Thermo).

All the extracts were tested in triplicate. Their effects were simultaneously compared with the negative control (H₂O₂) and positive control (resveratrol).

### Results

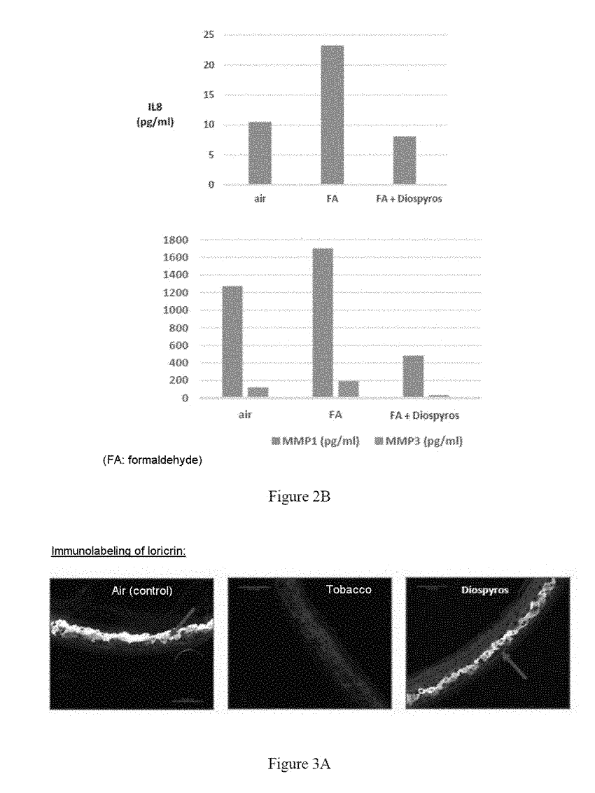

The effects of the negative and positive controls were validated. 100 μM of H₂O₂ induces a significant increase, by a factor of 9.4, in mitochondrial production of ROSs relative to the nontreated cells. Resveratrol at 1 μM considerably reduces the overproduction of mitochondrial ROSs induced by H₂O₂ in the keratinocytes (close to 1.3 times (p=0.013)) (FIG. 5).

The Sanicle extract significantly reduces the level of mitochondrial ROSs induced by H₂O₂, demonstrating an effective antioxidant activity (13% at 0.001%, p<0.05 and 38% at 0.005%, p<0.001).

The effects of the negative and positive controls were validated. 100 μM of H₂O₂ induces a significant increase of 7.4 times the cell ROS production level compared with the nontreated cells. Trolox at 10 μm considerably reduces the overproduction of cell ROSs induced by H₂O₂ in the keratinocytes (1.7 times, p<0.001).

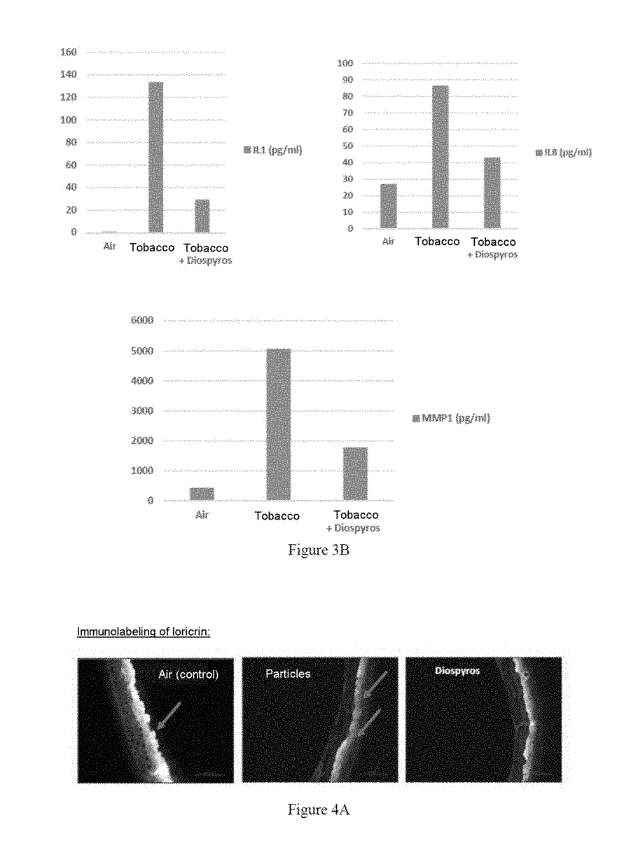

At the two doses, the Sanicle extract significantly reduces the production of cell ROSs induced by H₂O₂ (49% on average, p<0.001) (FIG. 6).

In the human keratinocytes, the exposure to H₂O₂ induced a non-significant trend toward a decrease in the cell SOD activity of close to 30% (p=0.075) compared with the nontreated cells, whereas it significantly increased the mitochondrial SOD activity by close to 211% (p=0.006) compared with the nontreated cells.

The antioxidant reference compound, 1 μM resveratrol, significantly increased the cell SOD activity by close to 40% (p=0.036) compared with the nontreated cells in response to H₂O₂ exposure. In addition, 1 μM resveratrol significantly reduced the activity of the mitochondrial SOD induced by H₂O₂ by close to 66% (p=0.021), maintaining an mtSOD level comparable with that of the nontreated cells.

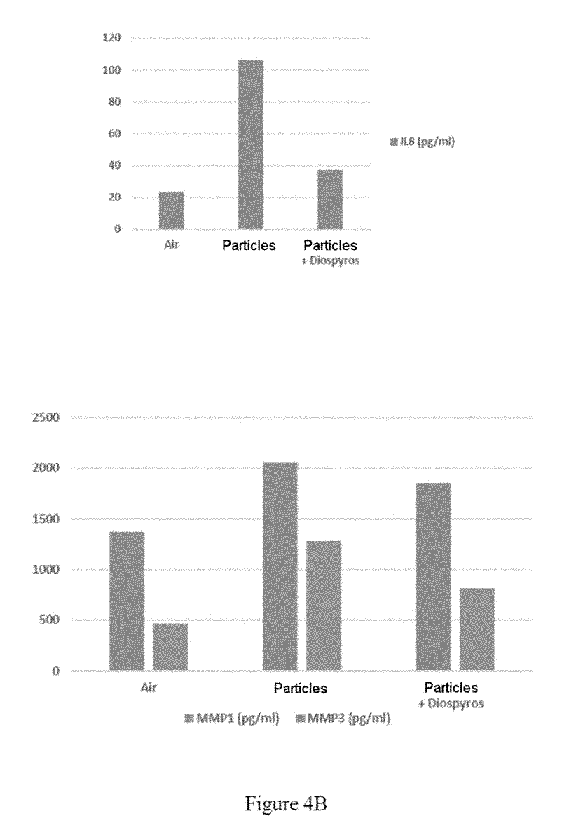

The treatment of the keratinocytes with the Sanicle extract at 0.001% results in a cell SOD activity that is comparable to that of the nontreated cells. At 0.005%, the Sanicle extract increased the cell SOD activity with regard to that induced by H₂O₂ compared with the nontreated cells by 76% (p=0.012), revealing an up-regulation of the cell antioxidant defenses in response to a cellular oxidative stress (FIG. 7).

The Sanicle extract at 0.001% restored the mitochondrial SOD activity comparable to that of the nontreated cells, considerably decreasing its induction by H₂O₂ by close to 80% (p=0.008), in a manner similar to the effect of the resveratrol at 1 μM.

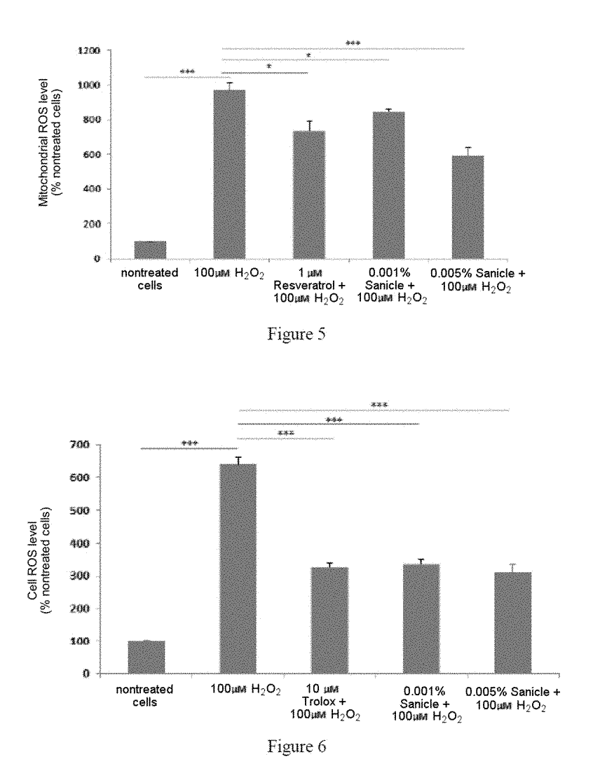

The Sanicle extract at 0.005% maintained the mitochondrial SOD activity induced by H₂O₂ (FIG. 8).

H₂O₂ at 100 μM significantly increased the total glutathione level by close to 2.9 times compared with the nontreated cells (p=0.044), revealing an up-regulation of glutathione in response to the oxidative stress induced by H₂O₂. The pre-incubation with resveratrol at 1 μM significantly reduced the total glutathione level after treatment with H₂O₂ (94%), restoring a level comparable to that of the nontreated cells (p=0.006).

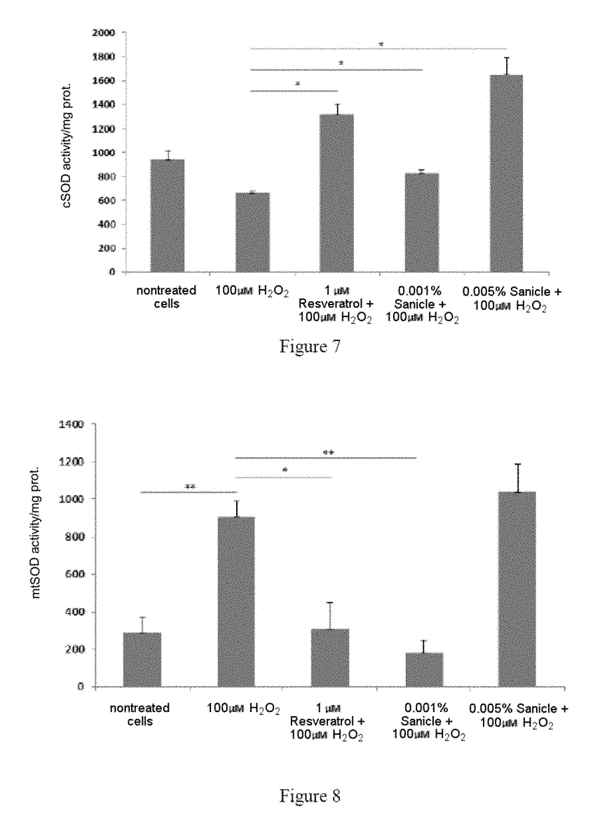

The Sanicle extract significantly decreased (like the resveratrol at 1 μM), the total glutathione level after treatment with H₂O₂ to a level comparable with that of the nontreated cells (FIG. 9).

### CONCLUSION

Exposure to H₂O₂ has been identified as an inducer of cell damage, compromising the oxidation-reduction equilibrium in primary human keratinocytes. The effects of H₂O₂ (100 μM, 1 hour) in the human keratinocytes are summarized in the table below.

Under the experimental conditions, H₂O₂ generated mitochondrial ROSs, resulting in the accumulation of intracellular ROSs. The increase in mitochondrial SOD activity and the increase in total glutathione level are a response to the H₂O₂-induced oxidative stress in order to counteract early increase in ROS production.

The increase in mitochondrial SOD and in GSH may be directly responsible for the observed reduction in cell SOD, through a retroactive loop, explaining the decrease in its activity where the exogenous H₂O₂ introduced by the treatment overcomes the activity of cell SOD, which converts O₂·⁻ to H₂O₂.

The effects of the Sanicle extract combined with exposure to H₂O₂ are summarized in the table below.

The Sanicle extract at 0.001% is an effective antioxidant with respect to mitochondrial and cell ROSs induced by H₂O₂. As a result, the Sanicle extract decreases the need to increase the SOD antioxidant enzymes and the glutathione, a nonenzymatic antioxidant system.

The Sanicle extract at 0.005% increases again the activity of the cell SOD, already solicited in the presence of H₂O₂, potentiating the first antioxidant defenses.

### EXAMPLE 3

The objective of this study is to present the anti-pollution effect of a Furcellaria lumbricalis extract at the concentration of 0.5%.

For this, pollution due to motor vehicle exhaust gases containing pollution elements common to many pollutant stresses, such as cigarette smoke, CO, NO and NO₂, SO₂, but also benzene, aldehydes, particles, volatile organic compounds (VOCs), polychlorobiphenyls (PCBs), heavy metals, etc. (Dupuis. E., 2006) was chosen.

The effect of this pollution was measured on human skin explants exposed to the pollution.

### Materials and Methods

### Biological Model

Human skin explants 12 mm in diameter, collected after an abdominal plasty operation on a 58-year-old Caucasian woman (Biopredic International, Rennes).

### Test and Reference Products

The extract tested is the commercial product Hydranov P from Codif, which, it is recalled, is an oligofurcellaran extract enriched with marine salts, obtained by depolymerization of a sulfated furcellaran (galactose and anhydrogalactose), then rehydrated with a solution enriched with marine salts.

The resulting extract was diluted to 0.5% in water.

### Test System

After reception of the explants, the latter are stabilized for 16 h at ambient temperature. Following this stabilization, a measurement of the moisturization is carried out before application of the products (T0). The skin fragments are then treated with a solution of a Furcellaria lumbricalis extract at 0.5% for 30 min. These explants are then exposed to the motor vehicle exhaust gases for 5 h. They are then incubated at the air/medium interface in a buffered long-term medium (Hepes) for 18 h at 20-22° C. After 18 h, the moisturization is measured (T1). The manipulation performed on these explants is represented schematically below:

Treatment with the Gas

The gas used in this test comes from a Renault diesel vehicle (1989) without a particle filter, and is collected using a standard airtight bag (brand SKC, ref. Cat. No. 236-005).

By way of indication, the composition of the gas is the following:

| Concentrations in μg/m³ | | Concentrations in μg/m³ | |
|---|---|---|---|
| Compound | Amount | Compound | Amount |
| 1-Propene-2-methyl | 1145.55 | Octane | 42.20 |
| Propene | 1988.62 | Heptane, 2,4-dimethyl | 34.91 |
| MEK | 184.26 | Nonane | 27.87 |
| Hexane | 53.66 | Decane | 23.90 |
| 1-Buten-3-one | 49.42 | Decane, 2,4,6-trimethyl | 13.36 |
| 2-Butenal | 46.34 | Undecane | 15.56 |
| Benzene | 140.12 | Ethylbenzene | 5.60 |
| Acetic acid | 181.60 | m + p Xylene | 17.11 |
| Toluene | 42.98 | Oxylene | 5.88 |
| 1-Octene | 21.28 | Acetone | 72.94 |

### Measurement of the Moisturization

a. Principle

The degree of moisturization of the skin was studied by means of a CM 825 MDD Corneometre0 (Courage & Khazaka). This instrument makes it possible to determine the degree of moisture of the outermost skin layers of the stratum corneum. The principle of action of the corneometer is based on the possibility for the detector, designed as a capacitor, to modify its capacity. The face of the measuring head, in contact with the skin, modifies its capacity according to the degree of moisture of the skin. These modifications are directly entered into a computer.

Various methods exist for measuring the degree of moisturization of the skin. Among them, corneometry is a conventional method in cosmetology. Based on the possibility for the detector to modify its capacity as a function of the degree of moisture of the outermost skin layers of the stratum corneum, the corneometer thus makes it possible to quantify, in vivo and directly on human beings, the effect of cosmetic or dermopharmaceutical products on the degree of moisturization of the stratum corneum.

b. Parameter Studied

The degree of moisturization is expressed in arbitrary units.

### An Increase in the Skin Moisturization Values Reflects a Moisturizing Effect of the Product.

### Statistical Analysis

The crude data are transferred and processed using the Excel software. The test used is the paired Student's t test. A difference between 2 groups is considered to be statistically significant if the p-value is less than 0.05, and is denoted *p<0.05.

### Results

Under the conditions of the test, the exposure to motor vehicle exhaust gases causes a clear decrease in moisturization (FIG. 1: Control+gas compared with Control−gas).

The Furcellaria lumbricalis extract strongly protected the skin against the drop in degree of moisturization (+78%) in the explants of human skin induced by pollution.

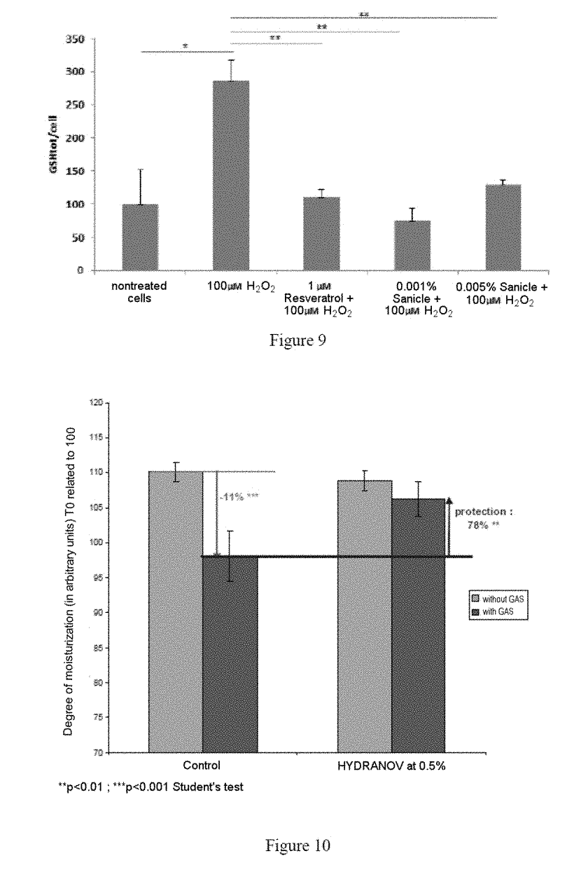

These results are illustrated in FIG. 10.

### CONCLUSION

Exposure to pollution causes a decrease in the degree of moisturization of the skin.

At the level of the human skin explants, the Furcellaria lumbricalis extract at 0.5% protects against the loss of moisturization induced by pollution.

### Examples of Compositions According to the Invention

ANTI-WRINKLE CREAM

| | % |
|---|---|
| CARBOMER | 0.30 |
| GLYCERIN | 2.00 |
| DIMETHICONE | 3.00 |
| ISONONYL ISONONANOATE | 8.00 |
| CETEARYL ALCOHOL & CETEARYL GLUCOSIDE | 3.50 |
| TOCOPHEROL | 0.06 |
| GLYCERYL STEARATE & PEG-100 STEARATE | 2.50 |
| DICAPRYLYL CARBONATE | 4.00 |
| SODIUM HYDROXIDE | 0.08 |
| TAPIOCA STARCH | 1.50 |
| BIOSACCHARIDE GUM-4 | 1.00 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| PRESERVATIVES | Q.S. |
| DISODIUM EDTA | 0.10 |
| FRAGRANCE | Q.S. |
| DEMINERALIZED WATER | Q.S. 100 |

SERUM

| | % |
|---|---|
| CETEARYL ETHYLHEXANOATE | 2.50 |
| STEARYL HEPTANOATE | 1.50 |
| CAPRYLIC/CAPRIC TRIGLYCERIDE | 3.00 |
| PEG-20 METHYL GLUCOSE SESQUISTEARATE | 2.00 |
| DIMETHICONE | 1.50 |
| TOCOPHEROL | 0.02 |
| ACRYLATES/C10-30 ALKYL ACRYLATE CROSSPOLYMER | 0.25 |
| ISONONYL ISONONANOATE | 3.00 |
| C12-15 ALKYL BENZOATE | 2.50 |
| HYDROXYETHYLCELLULOSE | 0.10 |
| GLYCERIN | 5.00 |
| BUTYLENE GLYCOL | 2.00 |
| ETHYLHEXYLGLYCERIN | 0.20 |
| DISODIUM EDTA | 0.05 |
| TROMETHAMINE | 0.30 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| PRESERVATIVES | Q.S. |
| FRAGRANCE | Q.S. |
| DEMINERALIZED WATER | Q.S. 100 |

O/W EMULSION

| | % |
|---|---|
| CETEARYL ALCOHOL & CETEARYL GLUCOSIDE | 3.50 |
| GLYCERYL STEARATE & PEG-100 STEARATE | 2.00 |
| STEARETH-21 | 0.50 |
| HYDROGENATED COCO-GLYCERIDES | 2.00 |
| SHEA BUTTER | 1.00 |
| CYCLOMETHICONE | 6.00 |
| BHT | 0.02 |
| CARBOMER | 0.20 |
| GLYCERIN | 5.00 |
| SODIUM HYDROXIDE | 0.04 |
| SILICA | 1.40 |
| TOCOPHERYL ACETATE | 0.20 |
| DIMETHICONE & DIMETHICONOL | 0.50 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| PRESERVATIVES | Q.S. |
| TETRASODIUM EDTA | 0.05 |
| FRAGRANCE | Q.S. |
| DEMINERALIZED WATER | Q.S. 100 |

SPF 20 DAY CREAM

| | % |
|---|---|
| ETHYLHEXYLGLYCERIN | 0.20 |
| DISODIUM EDTA | 0.10 |
| XANTHAN GUM | 0.20 |
| GLYCOLS | 5.00 |
| ACACIA SENEGAL GUM | 0.01 |
| BUTYLOCTYL SALICYLATE | 5.00 |
| SODIUM POLYACRYLATE | 0.50 |
| MICA | 0.10 |
| POTASSIUM CETYL PHOSPHATE | 0.50 |
| BUTYL METHOXYDIBENZOYLMETHANE | 3.00 |
| OCTOCRYLENE | 2.70 |
| HOMOSALATE | 10.00 |
| CETEARYL ALCOHOL & CETEARYL GLUCOSIDE | 3.50 |
| C12-15 ALKYL BENZOATE | 8.00 |
| TAPIOCA STARCH | 2.00 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| ALCOHOL | 3.00 |
| PRESERVATIVES | Q.S. |
| FRAGRANCE | Q.S. |
| DEMINERALIZED WATER | Q.S. 100 |

W/O EMULSION

| | % |
|---|---|
| PEG 30 DIPOLYHYDROXYSTEARATE | 4.00 |
| HYDROGENATED C16-C18 TRIGLYCERIDES | 5.00 |
| PEG-45/DODECYL GLYCOL COPOLYMER | 1.20 |
| C8-C10 TRIGLYCERIDES | 19.00 |
| LIQUID PETROLEUM JELLY | 8.00 |
| GLYCOLS | 10.00 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| PRESERVATIVES | Q.S. |
| FRAGRANCE | Q.S. |
| DEMINERALIZED WATER | Q.S. 100 |

AQUEOUS-ALCOHOLIC LOTION

| | % |
|---|---|
| GLYCOLS | 2.00 |
| BUFFER | 0.40 |
| SODIUM CHLORIDE | 1.00 |
| ETHANOL | 5.00 |
| LAMPSAN | 0.50 |
| FURCELLARIA LUMBRICALIS | 1.00 |
| DIOSPYROS MESPILIFORMIS | 0.50 |
| SANICULA EUROPAEA | 0.50 |
| TROMETHAMINE | 0.80 |
| PRESERVATIVES | Q.S. |
| SOLUBILIZING AGENT | Q.S. |
| FRAGRANCE | Q.S. |
| DEMINERALIZED WATER | Q.S. 100 |

1-15. (canceled)

16. A method of cosmetic treatment for protecting the skin against the harmful effects on the skin of atmospheric pollution and/or repairing skin manifestations induced by atmospheric pollution comprising the application to the skin of a human subject the combination of a Diospyros mespiliformis extract and at least one extract selected from a Sanicula europea extract, a Furcellaria lumbricalis extract and a Lampsana communis extract or of a composition comprising said combination.

17. The method as claimed in claim 16, wherein the atmospheric pollution results from the presence of interior or exterior pollutants chosen from oxidizing agents, carbon monoxide, hydrocarbons and solvents, sulfur dioxide, metals, formaldehyde, cigarette smoke, particulate matter or fine dust in suspension and electromagnetic waves.

18. The method as claimed in claim 16, wherein the application to the skin of the combination of a Diospyros mespiliformis extract and at least one extract selected from a Sanicula europea extract, a Furcellaria lumbricalis extract and a Lampsana communis extract or of a composition comprising said combination:
increases cohesion of the skin and stratum corneum and improves skin barrier function;
restores homeostasis of keratinocyte proliferation and differentiation processes;
restores the dermal matrix;
prevents and restore of the moisture of the skin;
improves cohesion of the skin cells;
improves cell communication;
restores the dermo-epidermal junction.

19. The method as claimed in claim 16, wherein the application to the skin of the combination of a Diospyros mespiliformis extract and at least one extract selected from a Sanicula europea extract, a Furcellaria lumbricalis extract and a Lampsana communis extract or of a composition comprising said combination prevents or removes wrinkles and fine lines, preserves radiance, increases moisture, regulates desquamation, and/or maintains or stimulates the suppleness of the skin subjected to pollution.

20. The method as claimed in claim 16, wherein the Diospyros mespiliformis extract is obtained by milling and extraction with aqueous and/or organic solvents of an aerial part of the plant.

21. The method as claimed in claim 16, wherein the composition comprises between 0.01 and 10% of Diospyros mespiliformis extract by weight.

22. The method as claimed in claim 16, wherein the composition comprises one or more agents selected from the group consisting of softeners, dyes, film-forming active agents, surfactants, fragrances, preservatives, emulsifiers, oils, glycols, vitamin E, and UV-screening agents.

23. The method as claimed in claim 16, wherein the composition is in the form of a gel, lotion, cream, emulsion, milk, or spray.

24. The method as claimed in claim 16, wherein said Sanicula europea extract is a water-soluble extract of the aerial parts of the plant.

25. The method as claimed in claim 16, wherein said Furcellaria lumbricalis extract comprises oligofurcellaran enriched with marine salts.

26. The method as claimed in claim 16, wherein said Lampsana communis extract is obtained by aqueous-glycolic extraction of the aerial part of the plant.

27. A method of preventing the effects of pollution on the skin in a human subject, comprising applying to the skin an appropriate amount of a Diospyros mespiliformis extract in combination with an extract selected from a Sanicula europea extract, a Furcellaria lumbricalis extract and a Lampsana communis extract or of a composition comprising said combinations.
